(19) United States
(12) Patent Application Publication
(10) Pub. No.: US 2026/0167917 A1
(43) Pub. Date: Jun. 18, 2026

(54) DEVICE AND METHOD FOR CHARACTERIZING CELLS SUBJECTED TO A PHYSICAL STRESS

(71) Applicants: CENTRE NATIONAL DE LA RECHERCHE SCIENTIFIQUE, Paris (FR); UNIVERSITE CLAUDE BERNARD LYON 1, Villeurbanne Cedex (FR); INSTITUT NATIONAL DES SCIENCES APPLIQUEES DE LYON, Villeurbanne (FR); ECOLE SUP CHIMIE PHYS ELECTRONIQ LYON (CPE), Villeurbanne (FR)

(72) Inventors: Edwin-Joffrey COURTIAL, Neyron (FR); Emma PETIOT, Villeurbanne (FR); Mehdi MALEKI, Villeurbanne (FR); Imen HALIMA, Saint-Priest (FR); Gabriel FROSSARD, Lyon (FR)

(73) Assignees: CENTRE NATIONAL DE LA RECHERCHE SCIENTIFIQUE, Paris (FR); UNIVERSITE CLAUDE BERNARD LYON 1, Villeurbanne Cedex (FR); INSTITUT NATIONAL DES SCIENCES APPLIQUEES DE LYON, Villeurbanne (FR); ECOLE SUP CHIMIE PHYS ELECTRONIQ LYON (CPE), Villeurbanne (FR)

(21) Appl. No.: 19/114,196

(22) PCT Filed: Sep. 22, 2023
(86) PCT No.: PCT/EP2023/076210

§ 371 Date: Feb. 20, 2026

(30) Foreign Application Priority Data
Sep. 23, 2022 (FR) FR2209653

(51) Int. Cl. C12M 1/42; C12M 3/06; G01N 15/1433; G06V 10/764; G06V 20/69

(52) U.S. Cl. CPC C12M 35/04; C12M 23/16; G01N 15/1433; G06V 10/764; G06V 20/698

(57) ABSTRACT

The present invention relates to a microfluidic device and to a method for applying at least one hydrodynamic stress of defined intensity and duration to cells in suspension, said cell being in motion in said device, and for characterizing the morphology and the physiological state of these cells during, and optionally after, the application of the hydrodynamic stress.

### FIELD OF THE INVENTION

The present invention relates to a microfluidic device and a method for applying at least one physical stress of determined intensity and duration to cells in suspension, and for characterizing the morphological and physiological state of the cells subjected to the application of this physical stress.

The present invention is therefore in the field of microfluidic devices and analytical cell biology.

### PRIOR ART

The currently known means of describing and characterizing cell morphology and physiology are based in particular on the use of cell markers. Morphology itself can be used as a marker of a cell's physiological state. However, on the one hand, these methods require appropriate markers specific to a physiological state and, on the other hand, they only characterize the physiological state of the cell as observed at the time of marking.

Furthermore, the efficiency of bioprocesses using animal cells, in particular such as bioprinting processes, bioproduction of cells for cell therapy, and cell-based processes such as the production of therapeutic proteins, viral vaccines or viral vectors, as well as all cell injection or harvesting processes, depends on the quality of the cells used in said bioprocesses.

Cell quality is generally related to physiological characteristics such as cell viability, that is, the proportion of living cells to cells engaged in cell death processes (apoptosis, necrosis, lysis), or cell function (ability to secrete molecules, ability to differentiate into cell subtypes in the case of stem cells).

The effectiveness of a bioprocess can therefore be strongly impacted by the alteration it produces on cell quality and therefore on cell physiology. One of the mechanisms that has a major impact on quality, and in particular cell viability, within a bioprocess is the application of hydrodynamic and mechanical stresses generated by the equipment used, such as a bioreactor, a system using an injection or sampling syringe, or a separation step. The interaction between a suspended fluid and a wall creates stress on the cells suspended in the fluid. Direct interaction of the cell with the wall produces a mechanical impact on the cell. Depending on the nature of the process and the techniques employed, the intensity and duration of a physical stress exerted on a cell can vary greatly. For example, a cell therapy process can generate a stress on the cells with an intensity of up to 5,000 Pa, and a bioprinting process can generate a stress with a duration of up to 30 ms.

It is therefore desirable to be able to characterize, in a simple and reproducible way, the physiological state of cells during and/or after the application of a physical stress, in particular by characterizing their morphology. In particular, this type of characterization is desirable for cells likely to be used in a bioprocess.

It is further desirable to characterize the physiological state of cells when they are subjected to physical stresses that reproduce the physical stress conditions applied to the cells during a particular bioprocess.

WO 2015/024690 “Apparatus and method for determining the mechanical properties of cells” relates to a method and apparatus for determining the mechanical properties of cells, and discloses the study of the deformation of cells subjected to shear stress. This document discloses a device comprising a single capillary channel wherein suspended cells are subjected to shear stress, and a method of measuring the shape of the cells as they flow through the device. This method uses binarized images. Measurements are taken in real time.

EP 3 796 212″Device for image-based cell classification, method therefor and use thereof” describes a real-time, marking-free cell sorting device. This device comprises a microfluidic network that aligns each cell along its major axis, and a classification unit comprising a neural network that sorts the cells based on images thereof.

WO 2019/006188 “Quantitative deformability cytometry: rapid, calibrated measurements of cell mechanical properties” describes a microfluidic device and a quantitative deformability cytometry (q-DC) method for quantitatively assessing the intrinsic mechanical properties of cells. The mechanical parameters determined are: elastic modulus E, cell fluidity P, transit time TT, migration time Tc, cell size Dcell and maximum stretch Emax, at a flow rate of 100 cells/s. Machine learning tools are used. The cells are subjected to calibrated shear stresses.

There is therefore a need for a device and method that not only characterize the mechanical properties of physically stressed cells, but further characterize their morphology and physiological state.

There is therefore a need for a device and method capable of generating at least one physical stress as generated during a bioprocess, the nature, intensity and duration of which are determined and controlled, in order to characterize the cells likely to undergo said stresses. “Physical stress” is understood to mean in particular any force exerted on the cells, capable in particular of generating cellular stress; such a physical stress may in particular be a mechanical impact or a hydrodynamic stress, such as an elongation, compression or shear stress.

### BRIEF DESCRIPTION OF THE INVENTION

The inventors have developed a device for applying at least one physical stress of determined intensity and duration to cells in suspension, and for characterizing the cells that have undergone said at least one physical stress.

More particularly, a device according to the invention is configured to:
apply at least one hydrodynamic stress of determined intensity and duration to at least one cell suspended and moving in said device,
and optionally apply at least one mechanical impact to said cell.

A device according to the invention is configured for characterization of said cell during application of said stress and optionally for characterization of said cell after application of said stress.

A device according to the invention comprises, on the one hand, a microfluidic circuit for applying at least one physical stress and, on the other hand, a means for characterizing the cells.

In a device according to the invention, said at least one physical stress is a stress selected from mechanical stresses, in particular mechanical impact, and/or hydrodynamic stresses, such as hydrodynamic compressive stress, hydrodynamic shear stress and/or hydrodynamic extensional stress. More particularly, in a device according to the invention, said at least one physical stress is a stress chosen from hydrodynamic stresses.

In a device according to the invention, said means of cell characterization is chosen from all known technical means of cell observation, in particular imaging or cytometry means. More particularly, in a device according to the invention, said means of cell characterization during application of the stress is chosen from all known technical means of cell observation, in particular imaging or cytometry means.

A device according to the invention comprises a microfluidic circuit comprising at least one segment configured for the application to cells in suspension of at least one physical stress, more particularly a hydrodynamic stress, this segment comprising at least: i) a main channel configured for the circulation of a fluid containing said cell in suspension, ii) a fluid inlet and a fluid outlet, iii) means for introducing and establishing a flow of said fluid inside said channel.

The first object of the invention is therefore a device for applying at least one physical stress, more particularly at least one hydrodynamic stress, of determined intensity and duration to at least one cell in suspension, and for characterizing said cell during and/or after said application, more particularly during and optionally after said application.

The second object of the invention is a method for applying at least one physical stress, more particularly at least one hydrodynamic stress, of determined intensity and duration to at least one cell in suspension, and for characterizing said cell during and/or after said application, more particularly during and optionally after said application.

Another object of the invention is a classification model, previously trained on a training dataset, for characterizing a cell and predicting its physiological state during and/or after the application of at least one physical stress, according to a device and method according to the invention. Such a classification model saves time and money and improves performance (in particular, it enables a large number of cell characterizations to be carried out in a short period of time), compared with existing tools on the market.

Finally, the invention relates to the use of a device, method or classification model according to the invention, to characterize a cell and possibly predict its physiological state during and/or after the application of at least one physical stress.

A device and method according to the invention have the advantage of generating at least one physical stress, the nature, intensity and duration of which are precisely determined and controlled, in order to characterize the cells that have undergone said stress.

A device and a method according to the invention have the advantage of enabling the application of a modulable sequence of at least one physical stress, in particular the repetition of the same stress and/or the combination of physical stresses of a different nature, the intensity and duration of each being precisely defined. Owing to this simple, precise tool, it is possible to reproduce the stresses applied during a bioprocess, and to monitor the state of at least one cell at high speed.

A device and method according to the invention also have the advantage of being able to predict the physiological state of cells during and/or after they have been subjected to physical stress. This prediction can be made during and up to several days after the application of these physical stresses.

Finally, a device and a method according to the invention have the advantage of defining the capacity of a cell to undergo the application of at least one determined physical stress while maintaining a morphology and a physiological state compatible with the requirements of said bioprocess. Owing to a device and a method according to the invention, it is possible to define, for given cells, a map of their resistance capacities to at least one physical stress.

“Capacity” refers to a cell's ability to withstand at least one physical stress of specified intensity and duration, without alteration of its physiological state. “Alteration of its physiological state” refers in particular to differentiation, or the transition from a viable state to a state of lysis, necrosis or apoptosis.

In particular, the use of a device and method according to the invention makes it possible to optimize bioprocesses, and in particular the associated physical stresses, in order to define optimal operating conditions and adapt said stresses to the cells of interest. These conditions focus, for example, on bioprocess parameters (temperature, flow rate, rotation speed) and the suspending fluid (viscosity, osmolarity). It is indeed desirable to optimize the stresses associated with the bioprocesses, in order to adapt said stresses to the data obtained during characterization and prediction of the physiological state of cells subjected to these physical stresses.

### DETAILED DESCRIPTION OF THE INVENTION

According to a first object, the invention relates to a device for applying at least one physical stress, more particularly at least one hydrodynamic stress, to at least one cell in suspension and for characterizing said cell during and/or after said application, the device comprising:
a microfluidic circuit comprising at least one segment, said segment comprising at least: i) a main channel configured for the circulation of a fluid containing said cell in suspension, ii) a fluid inlet and a fluid outlet, iii) means for introducing and establishing a flow of said fluid within said channel, and
means for characterizing said cell;
said device according to the invention being characterized in that said at least one segment is configured for the application to said cell of at least one physical stress selected from:
a mechanical impact,
a hydrodynamic compressive stress,
a hydrodynamic shear stress, and
a hydrodynamic extensional stress.

In the context of the present invention, the term “for” as in “for the application of physical stress” means “configured for the application of physical stress”. When an interval or range of values is indicated, the bounds cited are considered to be part of said range of values.

The term “microfluidic circuit” refers to a circuit designed to handle small volumes of fluid (10⁻¹⁸ to 10⁻³ liters) in channels with diameters ranging from 5 μm to 3000 μm.

“Fluid” is understood to mean a deformable medium suitable for circulation in a device according to the invention and for cell suspension.

Preferably, in a device according to the invention, said fluid is chosen from Newtonian fluids, that is, whose viscosity does not vary based on shear stress. Said fluid has at least one of the following properties:
its Newtonian viscosity is between 1 and 1000 mPa·s, preferably between 1 and 100 mPa·s,
its pseudoplasticity index is between 0 and 1, preferably 1 mPa·s,
it is compatible with cell survival under unstressed conditions, with an osmolarity of between 250 and 410 mOsmol,
its pH is physiological under ambient air handling conditions, said pH is therefore between 6.5 and 7.8 and
it comprises nutrients that can be assimilated by the cells in suspension, such as glucose, glutamine or a nutrient composition such as a culture medium.

More particularly, in a device and method according to the invention, the fluid for the cell suspension is preferably chosen from Newtonian fluids, and in particular from:
physiological buffers, usually used for cell suspension, such as PBS, HBSS; these buffers may be supplemented with nutrient compounds such as 0.5 to 6 g/l glucose solution and 2 to 4 mM glutamine solution,
cell culture media, such as DMEM, EMEM, alpha-MEM,
Cell separation solutions, such as Ficoll and sucrose solution,
Low-molar-weight polymer solutions.

When applying said at least one physical stress, the cell concentration is preferably less than 100 million cells per mL, preferably between 0.01 and 10 million cells per mL. In other words, the cell volume preferably represents at most 30% of the total volume of the suspension fluid.

In a device according to the invention, the reference fluid and the fluid wherein the cells are suspended are circulated in a stable, pulsation-free flow. Said device is therefore configured to apply at least one stress to at least one cell in suspension and moving in said device. The device is preferably designed to conduct cells to a microfluidic chip along a defined streamline.

In a device and method according to the invention, the intensity of the stress, also referred to as “stress level” or “stress” in the figures, and the residence time under stress, also referred to as “stress duration” or “time” in the figures, take place in a controlled manner. The capture and release of suspended cells also take place in a controlled manner.

The pump(s) control the flow rate and flow pattern. The pumps are preferably ultra-high pressure, up to 1.37×10⁵ kPa.

The circulation of a fluid within a device according to the invention is initiated and maintained by means of any suitable apparatus well known to a person skilled in the art, such as a pump.

In a device according to the invention, the cross-section of the channels can be constant or variable, or conical, for example in the form of nozzles. The presence of an increasing or decreasing conical section at the end of a channel imposes an additional stress on the cells in terms of capture and controlled release.

When a fluid containing cells in suspension is circulated in the microfluidic circuit, current lines are generated to control the trajectory of the cells. The cells to be characterized are positioned on one of the current lines enabling control of the stress intensity.

The preferential diameter of the main channel is between 10 and 3000 μm.

The channels are made of a material suitable for the circulation of a fluid subjected to a pressure of between 10⁻³ and 106 kPa, and preferably between 1 and 10⁴ kPa, and/or of a material suitable for the circulation of a fluid with a viscosity of between 1 and 2000 mPa·s.

The capacity of cells depends on their origin (clone, species, organ), their culture mode (number of doublings in culture, culture medium, environmental condition of the culture, that is, whether or not stirred, temperature, pH) and their collection mode (trypsination, mechanical harvesting). For example, the type of cell culture, in particular 2D or 3D, their adherent culture or culture in suspension, and the type of culture medium used influence cell capacity

In a particular embodiment of a device according to the invention, the microfluidic circuit consists of a set of channels made of a suitable material, in particular selected from PEEK (PolyEtherEtherKetone), PVC (PolyVinyl Chloride), PTFE (PolyTetraFluoroEthylene), FEP (Fluorinated ethylene polypropylene), PDMS (PolyDimethylSiloxane) or steel. In another embodiment, the microfluidic circuit consists of a microfluidic chip made of a material selected from PDMS, polyacrylate, SEBS (StyreneEthyleneButyleneStyrene), glass, polycarbonate or ceramic.

In particular embodiments, a device according to the invention may comprise channels arranged in series and/or channels arranged in parallel.

Fluid circulation in a device according to the invention is characterized by at least one of the following parameters:
a fluid flow rate, in the channel considered for characterizing the physical stress applied to the cells, of between 0 and 5 ml/min, preferably between 0 and 1 ml/min, and/or
a fluid flow rate of between 5 μm/s and 300 m/s, and/or
a throughput of analyzed cells that can exceed 1000 cells/minute when the analysis is performed with software.

The total duration of cell presence in said channel is preferably between 1 μs and 10,000 s, preferably between 1 μs and 100 s, preferably between 1 us and 1 s, preferably between 10 μs and 100 ms.

“Suspension cell” is understood to mean any type of cell, animal or plant, prokaryotic or eukaryotic. Depending on the diameter of the channels and the magnification of the lens used for image analysis, a device according to the invention can be used to characterize bacterial, algal or fungal cells, for example.

The invention particularly relates to a device for applying at least one physical stress and characterizing at least one cell in suspension for at least one of the following aspects:
the size of the cell,
the shape of the cell, in particular the sphericity of the cell
the appearance of the outer membrane,
the appearance of cytoplasm, particularly its granularity
the presence of at least one marker on the cell surface,
the protein content of the cell, and
the nucleic acid content of the cell, in particular the quantity of DNA, the quantity of RNA, the nucleotide sequence of one or more nucleic acids, whether DNA or RNA.

“Cell characterization” is understood to mean the definition of at least one characteristic of said cell. If more than one cell characteristic is defined, the characterization of said cell comprises the definition of the combination of said characteristics.

Cell characterization leads to determination of the physiological state of the cells during or after application of said at least one stress. Physiology studies the role, function and mechanical, physical and biochemical organization of cells and their components, particularly cell organelles. Physiology also studies the interactions between a cell and its environment. Determining the physiological state of cells comprises, in particular, the state of differentiation and/or the determination of nutritional, proliferative and relational functions, such as mobility and sensory functions.

This physiological state is preferably chosen from the following: living cell, dead cell, lysed cell, necrotic cell, apoptotic cell, differentiated cell or non-differentiated cell, pathological cell or healthy cell.

The relationship between the physiological state and the various aspects of characterization are known to the skilled person.

The physiological state of the cells after the application of at least one physical stress can further be compared with the physiological state of the cells before the application of said physical stress.

For the purposes of the present invention, the ability of cells to undergo at least one physical stress of specified intensity and duration, while maintaining a physiological state compatible with subsequent use, is defined as the cells' “capacity”.

According to a particular aspect, the invention relates in particular to a device configured for applying at least one physical compressive stress to said cell, said device comprising at least one segment of type (A) comprising, or consisting of, a first channel joined, at the same level, by two channels, each forming an angle of between 30 and 150 degrees with said first channel, this angle also being referred to as the “flow focusing angle”.

“Hydrodynamic compressive stress” is understood to mean the application of balanced forces toward the interior of the cells, also known as “flow focusing”. In a device and method according to the invention, the intensity of the compressive stress applied to the cells is between 10⁻³ and 10³ kPa, preferably between 10⁻³ and 10² kPa, preferably between 103 and 10 kPa. FIG. 1 schematically shows an example of a Type A segment.

According to another particular aspect, the invention particularly relates to a device configured for the application to said cell of at least one hydrodynamic shear stress, said device comprising at least one type B segment comprising, or consisting of, a channel of diameter between 10 and 2000 μm, preferably between 20 and 200 μm.

“Hydrodynamic shear stress” is understood to mean a mechanical stress applied parallel or tangentially to the face of a material. In a device and method according to the invention, the intensity of the shear stress applied to the cells is between 10⁻³ and 10⁵ kPa, preferably between 10⁻³ and 10⁴ kPa, preferably between 0.1 and 10³ kPa.

A hydrodynamic shear stress is applied in particular during cell circulation in a capillary channel, the diameter of which is preferably between 10 and 2000 μm, preferably between 20 and 200 μm. FIG. 1 schematically shows an example of a Type B segment.

According to another particular aspect, the invention particularly relates to a device configured for applying at least one physical extension stress to said cell, said device comprising at least one C-type segment comprising, or consisting of, i) a channel of increasing or decreasing cross-section, or ii) a first channel joined by a second wherein the fluid flows in a different, preferably opposite, direction to that of the first channel.

“Hydrodynamic extensional stress” is understood to refer to the application of balanced forces toward the outside of the cells, or elongation stress. In a device and method according to the invention, the intensity of the extensional stress applied to the cells is between 10⁻³ and 10³ kPa, preferably between 10⁻³ and 10² kPa, preferably between 10⁻³ and 10 kPa. FIG. 1 schematically shows an example of a Type C segment.

According to another particular aspect, the invention particularly relates to a device configured for the application to said cell of at least one mechanical impact, said device comprising at least one type D segment comprising, or consisting of, a first channel within which the cell path line encounters an obstacle, such as in particular the wall of a second channel. FIG. 1 schematically shows an example of a Type D segment.

“Mechanical impact” is understood to mean any type of mechanical impact, such as an impact with a wall or inertial collision with the surface. In a device and method according to the invention, the intensity of the mechanical impact applied to the cells is between 1 and 300 m/s, preferably between 1 and 100 m/s, preferably between 1 and 10 m/s. The duration of the impact time is preferably less than 1 μs.

More particularly, a device according to the invention for applying at least one physical stress and characterizing at least one cell in suspension comprises a microfluidic circuit comprising or consisting of:
at least one type A segment, and/or
at least one type B segment, and/or
at least one type C segment, and/or
at least one type D segment,
   said segments being combined with one another.

Even more particularly, a device according to the invention for applying at least one physical stress and characterizing at least one cell in suspension comprises a microfluidic circuit comprising or consisting of:
at least one type A segment, and/or
at least one type B segment, and/or
at least one type C segment,
and optionally at least one type D segment,
   said segments being combined with one another.

A device according to the invention is in particular designed to apply a sequence comprising one or more iterations of a physical stress of the same type, at a determined frequency and intensity, and/or to apply a sequence of several physical stresses of different types, at a determined frequency and intensity.

“Application of at least one physical stress of determined intensity and duration” is understood to mean:
at least one application of a particular physical stress of determined intensity and duration, optionally followed by the application of one, two, three, four or more repetitions of the application of said particular physical stress, and/or
at least one application of a sequence of at least two specific physical stresses of determined intensity and duration, optionally followed by the application of one, two, three, four or more sequences of at least two physical stresses, and/or
the application of any type of physical stress sequence as described in this application.

According to another particular aspect, the invention in particular relates to a device configured for the application to said cell of at least one sequence comprising, or consisting of, at least two successive physical stresses of different natures. The sequence can be repeated once, twice, three times or more.

According to another particular aspect, the invention in particular relates to a device configured for the application to said cell of at least one sequence comprising, or consisting of, at least two successive physical stresses of the same nature. According to this particular aspect, the device is configured for the application to said cell of a physical stress repeated at least 1 time, at least twice, at least 3, 4, 5, 6, 7, 8, 9, 10 times, or even more.

An example of this type of device according to the invention is shown in FIG. 2, which shows a device designed to apply a large number of compressive stresses to cells, separated by segments designed to ensure that the cells are not subjected to stress. These physical stress sequences can be considered equivalent to the application of dynamic cell deformation. These are repetitions of the same stress sequence.

More particularly, a device according to the invention is characterized in that the total intensity of said at least one physical stress applied to the cell is between 10⁻³ kPa and 10⁵ kPa, preferably between 10⁻³ kPa and 10⁴ kPa, preferably between 0.1 kPa and 10³ kPa, preferably between 1 kPa and 10² kPa.

More particularly, furthermore, a device according to the invention is characterized in that the total duration of the application of said at least one physical stress is between 1 μs and 10000 s, preferably 1 μs and 100 s, preferably between 1 μs and 1 s, preferably 10 μs and 100 ms.

In a microfluidic device according to the invention, the characterization of said at least one cell in suspension is carried out during the application to said cell of at least one physical stress and/or after the application to said cell of at least one physical stress. According to a first embodiment, the characterization of said at least one cell in suspension is carried out during the application of at least one physical stress to said cell.

According to another embodiment, a device according to the invention is characterized in that the characterization of said cell takes place after the application of said at least one stress, this characterization being carried out during a period of between 0 and 120 days, preferably between 0 and 30 days, preferably between 0 and 1 day after the application of said at least one physical stress.

More particularly, a device according to the invention is characterized in that the characterization of said cell after the application of said at least one stress comprises, or consists of, at least one discrete characterization or at least one characterization carried out over a duration of between 1 μs and 10000 s, preferably 1 μs and 100 s, preferably between 1 μs and 1 s, preferably 10 μs and 100 ms.

More particularly, a device according to the invention is characterized in that the means for characterizing said at least one cell is chosen from: a cytometer, a microscope, a means for analyzing the protein content of the cell, a means for analyzing and sequencing the nucleic acids of said cell, and an image and/or electrical signal capture means, combined with a signal analysis means. For example, a device according to the invention may comprise a microelectrode or a photodiode.

More particularly, a device according to the invention is characterized in that said signal and/or image analysis means comprises, or consists of, a central computer unit comprising software means adapted for signal and/or image analysis.

Even more particularly, a device according to the invention comprising signal and/or image analysis means further comprises a first and/or second classification model. The presence of at least a first and/or second classification model has the advantage of speeding up the image analysis process and enabling real-time analysis.

“Classification model” is understood to mean a previously trained machine learning algorithm, in particular a supervised learning algorithm, as well as a training dataset for training said algorithm, and an evaluation dataset. A classification model can consist of a computer program, which can be written in any suitable computer language known to a person skilled in the art. Said computer program is capable of being implemented on a computer to generate a technical result. Examples of these technical results are described below.

The training dataset can comprise a training set and a model test set. In this way, the model can be tested on the test base, and the test set can be used to determine whether or not model learning is satisfactory. The training set and the test set can be different. Alternatively, the test set can correspond to part of the training set.

Even more particularly, a device according to the invention comprising image analysis means further comprises a first classification model, previously trained with a training dataset, and comprising a supervised, unsupervised or semi-supervised machine learning algorithm. Said first classification model is adapted to predict the physiological state of a given cell based on at least one characteristic of said cell.

In a particular embodiment of the first classification model, the input data are images with objects. The output data are objects with a label: a percentage of membership in a specific class. The training algorithm comprises at least 10 epochs, the loss calculation is cross-entropy and the optimizer is Adam (improved gradient descent). The model is transfer learning with YOLO, the nature of the network is a supervised model (1733 cell images/1733 annotations). The model preferably comprises 106 convolutional layers. The functions performed in a neuron are: convolution, addition, softmax, up sampling. Neuron/layer connections are made. The training dataset can comprise a multitude of data pairs, each of the data pairs comprising a first data item representing at least one characteristic of said cell and a second data item representing a physiological state of said cell.

The training dataset can be built up in advance from data obtained in the laboratory by analyzing the characteristics of cells whose physiological state has been determined.

In particular, said first classification model can be implemented on a computer to generate a technical result consisting, for example, of a classification of a cell according to its characteristics.

Said first classification model is used to generate a three-dimensional diagram, representing, for example, for a given cell:
the intensity of the physical stress applied to the cells, expressed in Pa,
the duration of the physical stress applied to the cells, or residence time, or “time”, expressed in s,
the physiological state of the cells, that is, cell viability, necrosis, apoptosis or lysis, expressed as a percentage, or differentiated or undifferentiated nature.

The first classification model is considered to have reached a satisfactory level of learning on all the profiles in the test set if, in particular, the classification reaches a minimum FI score of 70%.

Even more particularly, when a device according to the invention comprises an image analysis means, said image analysis means further comprises a second classification model, previously trained with a training dataset, and comprising a supervised, unsupervised or semi-supervised automatic learning algorithm, said second classification model being adapted for detecting and monitoring the deformation of a given cell, in response to at least one physical stress.

In particular, said first classification model can be implemented on a computer to generate a technical result consisting, for example, in monitoring the morphological evolution of a cell according to different applications of physical stresses.

Preferably, said second classification model uses at least one neural network whose functions are as follows:
i) Locate and isolate the cell from said image
ii) Validate the presence of a single cell
iii) Improve image quality, then process the image by applying a mask to the cell and determining its outline,
iv) Position and measure the major and minor axes of the ellipse describing the cell contour. Deformation is defined as the ratio of the major axis to the minor axis.

According to a particular embodiment, the second classification model comprises: input data 440 grayscale-adjusted sliced cell images, output data: segmentation binary mask. The training data comprise 420 training images. For the training algorithm at least 10 epochs are required, the loss calculation is cross-entropy and the optimizer is Adam (improved gradient descent). The nature of the network is U-Net. The number of layers is: 5 contraction layers, 5 expansion layers, for a total of 10. The functions performed in a neuron are: 2D convolution between image and filter, that is, compressing the image, extracting the feature vector containing the object of interest and decompressing the image. The neuron/layer connections are characterized by the fact that the layers are composed of two convolutions, both followed by activation functions (ReLU).

An example of cell localization and isolation is shown in FIG. 3. An example of how to position and measure the major and minor axes of the ellipse is shown in FIG. 4.

The second classification model is considered to have reached a satisfactory level of learning on all the profiles in the test set if the classification achieves a minimum F1 score of 65%, preferably at least 80%.

According to a second object, the invention relates to a method for applying at least one physical stress of determined intensity and duration to at least one cell in suspension, and for characterizing said cell after said application, the method comprising the following steps:
a) depositing and circulating a fluid containing said at least one cell in suspension in a microfluidic circuit comprising at least one segment, said segment comprising at least: i) a main channel configured for the circulation of a fluid containing said cell, ii) a fluid inlet and a fluid outlet, iii) means for introducing and establishing a flow of said fluid within said channel, and
b) characterizing said cell after application of said at least one stress, a method according to the invention is characterized in that said at least one segment is configured for the application to said cell of at least one physical stress selected from:
a mechanical impact,
a hydrodynamic compressive stress,
a hydrodynamic shear stress, and
a hydrodynamic extensional stress.

A method according to the invention particularly relates to the application of a physical stress and the characterization of at least one cell in suspension, said characterization concerning at least one of the following aspects: the size, the shape, the appearance of the outer membrane, the appearance of the cytoplasm, the presence of at least one marker on the cell surface, the protein content of the cell and the nucleic acid content of the cell.

More particularly, the invention relates to a method according to the invention comprising the application of at least one physical stress to at least one cell in suspension, said physical stress being characterized in that the cell is subjected to:
at least one mechanical impact with an intensity of between 1 and 300 m/s, preferably between 1 and 100 m/s, preferably between 1 and 10 m/s and/or for a duration of less than 1 μs, and/or
at least one shear stress of intensity between 10⁻³ kPa and 10⁵ kPa, preferably between 10⁻³ kPa and 10⁴ kPa, preferably between 0.1 kPa and 10³ kPa, preferably between 1 kPa and 10² kPa and/or for a duration between 1 us and 10,000 s, preferably between 1 μs and 100 s, preferably between 10 us and 1 s, and/or
at least one compressive stress of intensity between 10⁻³ kPa and 10⁵ kPa, preferably between 10⁻³ kPa and 10⁴ kPa, preferably between 0.1 kPa and 10³ kPa, preferably between 1 kPa and 10² kPa and/or for a duration between 1 us and 10 s, preferably between 1 μs and 1 s, preferably between 10 μs and 10 ms, and/or
at least one extensional stress of intensity between 10⁻³ kPa and 10⁵ kPa, preferably between 10³ kPa and 10⁴ kPa, preferably between 0.1 kPa and 10³ kPa, preferably between 1 kPa and 10² kPa and/or for a duration between 1 us and 10 s, preferably between 1 μs and 1 s, preferably between 10 μs and 10 ms.

More particularly, in a method according to the invention, the parameters of i) fluid flow rate and ii) fluid viscosity are chosen in order to reproduce the stress intensities and residence time reproducing the stresses that are experienced by the cells during a particular bioprocess. As the fluid flows through the device, a streamline is generated. As the fluid flows through the device, the cell follows the streamline developed by the fluid's movement and its interaction with the channel geometry. By calculating or measuring the hydrodynamic stress on this line and the movement speed of the cell, it is possible to deduce the stress and residence time for the cell.

Preferably, in a method according to the invention, the fluid flow rate is between 10⁻³ and 10 ml/min, preferably between 10⁻³ and 1 ml/min. Preferably, in a method according to the invention, the residence time of the cell is between 1 μs and 10,000 s, preferably between 1 μs and 1 s, preferably between 10 μs and 100 ms.

More particularly, the invention relates to a method according to the invention comprising the application of at least one physical stress to at least one cell in suspension, followed by the characterization of said cell, the method further comprising a step of predicting the physiological state of a cell by a previously trained first classification model, on the basis of the characteristics determined in step b).

More particularly, the invention also relates to a method according to the invention comprising the application of at least one physical stress to at least one cell in suspension, followed by the characterization of said cell, the method further comprising a step of predicting the physiological state of a cell by a previously trained first classification model, on the basis of the characteristics determined in step b), said first classification model comprising: a machine learning algorithm, a supervised, semi-supervised or unsupervised learning neural network, previously trained with a training dataset.

More particularly, the invention also relates to a method according to the invention further comprising a step of monitoring the deformation of a cell at different times during its residence in said microfluidic channel, by a second previously trained classification model, based on the characteristics determined in step b).

According to a third aspect, the invention also relates to a classification model, previously trained on a training dataset to predict, in a method according to the invention, a physiological state of a cell after the application of at least one physical stress.

According to a fourth aspect, the invention relates to the use of a device or method according to the invention, or a classification model according to the invention, to characterize cells of the following type: prokaryotic cell, eukaryotic cell, animal cell, plant cell, human cell, stem cell, epithelial cell, fibroblast, blood cell, genetically modified cell or synthetic cell mimic.

More particularly, according to this fourth aspect, the invention relates to the use of a device or method according to the invention, or a classification model according to the invention, to determine the capacity of a cell.

Even more particularly according to this fourth aspect, the invention relatives to the use of a device or method according to the invention, or of a classification model according to the invention, for the definition of at least one parameter of a bioprocess.

Even more particularly, the invention relates to the use of a device or method according to the invention, or of a classification model according to the invention for defining at least one parameter of a bioprocess chosen from: bioprinting, cell therapy and bioproduction.

The present invention will be better understood on reading the following examples, which are given to illustrate the invention and not to limit its scope. In particular, it is possible to imagine variants of the invention that comprise only a selection of the features disclosed hereinafter in isolation from the other features disclosed, if this selection of features is sufficient to confer a technical benefit or to differentiate the invention with respect to the prior art.

### FIGURES

FIG. 1 is a schematic depiction of examples of segments A, B, C and D.

FIG. 2 is a schematic depiction of an example of a device wherein several types of physical stress are applied to the cells, in this case a sequence of shear and elongation stresses. The lower graph depicts a magnification of the upper graph. The cell is subjected to repeated stress.

This type of stress can also be defined as dynamic deformation, or oscillatory deformation.

FIG. 3 shows the steps involved in isolating a cell.

FIG. 4 shows the measurement of the minor and major axes of the ellipse.

FIG. 5, in Example 2, shows the viability state of AD-MSC human mesenchymal stem cells after the application of shear stresses of varying intensity and duration. The percentage of cells in a particular physiological state is given as a function of the duration of application (time), expressed in seconds, and the intensity (stress), expressed in Pa, of the shear stress. FIG. 5 shows the percentage of viable cells (frame A), lysed cells (frame B), necrotic cells (frame C) and apoptotic cells (frame D).

FIG. 6, in example 3, shows the viability state of fibroblast cells after the application of shear stress. Fibroblasts are characterized after the application of shear stresses of varying intensity and duration. The percentage of cells in a particular physiological state is given as a function of the duration of application (time), expressed in seconds, and the intensity (stress), expressed in Pa, of the shear stress. FIG. 6 shows the percentage of viable cells (frame A), lysed cells (frame B), necrotic cells (frame C) and apoptotic cells (frame D).

FIG. 7, in Example 4, shows the proportion of non-differentiated AD-MSC stem cells (white circle) and differentiated cells (black circle) after application of a shear stress, as a function of the duration of application (time), expressed in seconds, and the intensity (stress), expressed in Pa of the shear stress.

FIG. 8, in Example 5, shows the viability state of HEK293T cells after application of elongation stress. HEK293T cells are characterized after the application of elongation stresses of varying intensity and duration. The percentage of cells in a particular physiological state is given as a function of the duration of application (time), expressed in seconds, and the intensity (stress), expressed in Pa, of the elongation stress. FIG. 8 thus shows the percentage of cells in apoptosis (frame A), viable cells (frame B), necrotic cells (frame C) or lysed cells (frame D).

FIG. 9, in Example 6, shows the viability state of fibroblast cells after shear stress has been applied, viability being measured by cytometry (histogram bars) or trypan blue staining (black circles).

FIG. 10, in Example 7, shows the viability state of HEK293T cells after the application of shear stresses of varying intensity and duration. The percentage of viable cells, measured by trypan blue staining, is given as a function of the duration of application (time), expressed in seconds, and the intensity (stress), expressed in Pa, of the shear stress. The percentage of viable cells is expressed after a small number of passages (HEK293T P07, white triangles) or after a larger number of passages (HEK293T P18, black circles).

FIG. 11, in Example 8, shows the viability state of fibroblast cells after the application of shear stresses of varying intensity and duration, according to two series of measurements carried out on the same cell sample (P06), depicted by white triangles and black circles, respectively. P06 designates the 6th generation of cell culture passages.

FIG. 12, in Example 9, shows in three dimensions and on a logarithmic scale the percentage of cell viability as a function of hydrodynamic stress intensity and residence time.

FIG. 13, in Example 9, shows in two dimensions the percentage viability of fibroblasts on the y-axis, as a function of the intensity of a hydrodynamic shear stress, on the x-axis, and residence time, depicted according to the dot pattern.

FIG. 14, in Example 9, shows in two dimensions the percentage of cell viability as a function of hydrodynamic stress intensity and residence time.

FIG. 15, in Example 10, is a histogram depicting cell viability as a function of shear stress intensity, in kPa. For each stress intensity, the viability value was measured after several experiments.

FIG. 16, in Example 10, is a pictorial of the standard deviation (light bar), standard error (black bar) and coefficient of variation (gray bar) of the various measurements, as a function of the shear stress intensity, in kPa.

FIG. 17, in Example 10, is a histogram depicting the percentage of cell viability as a function of shear stress intensity, in kPa.

FIG. 18, in Example 11, shows: i) on the left, the percentage of cell viability of different cell types as a function of the intensity of hydrodynamic stress applied, in kPa, ii) on the right, for each of the cells studied, the percentage of cell viability as a function of the hydrodynamic stress applied.

FIG. 19, in Example 12, shows the percentage of cell viability as a function of the intensity of shear stress applied, in kPa. The symbols P07 (round), P08 (square), P09 (triangle) and P10 (star) represent the percentage of cell viability of cells defined according to the number of cell passages in culture.

FIG. 20, in Example 12, shows the percentage of cell viability as a function of the intensity of shear stress applied, in kPa. P09, P10 and P11 represent the percentage of cell viability of cells defined according to the number of cell passages in culture.

FIG. 21, in Example 13, shows the succession of the same hydrodynamic stress.

FIG. 22, in Example 13, shows the percentage of cell viability as a function of the intensity of hydrodynamic stress applied, in kPa.

FIG. 23, in Example 13, shows the percentage of cell viability as a function of the number of stress application cycles, for a stress of 0.2 kPa.

FIG. 24, in Example 14, shows images of the steps involved in locating and isolating a cell from an original image.

FIG. 25, in Example 15, shows the steps involved in detecting the axes of a cell from an original image.

FIG. 26, in Example 15, shows the deformability of a cell based on its initial diameter, under shear stress.

FIG. 27, in Example 15, shows the deformability of a cell based on its initial diameter, under the effect of a mechanical impact stress.

FIG. 28, in Example 16, schematically shows an “autoencoder” neural network with an example of the transformation of an initial image (input) into a final image (output) after processing by this network.

FIG. 29, in Example 16, shows the differences in image processing by the supervised network (U-net) of an initial image pre-processed or not by an unsupervised network (autoencoder); line 1 shows the results without application of an autoencoder, line 2 shows the results with application of a “variational autoencoder”, line 3 shows the results with application of a “denoising autoencoder”.

FIGS. 30A and 30B, in Example 17, show the contour results after application of the various neural networks and the associated metrics, then the difference in deformability calculation with respect to a contour done manually (Control at 0).

FIG. 31, in Example 18, shows a classification result of three cells with, from left to right, a necrotic cell, an apoptotic cell and a living cell.

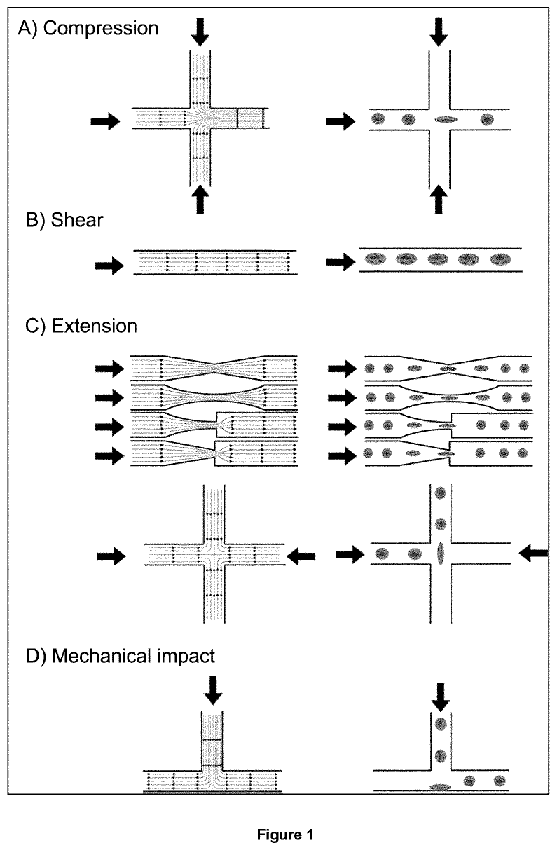

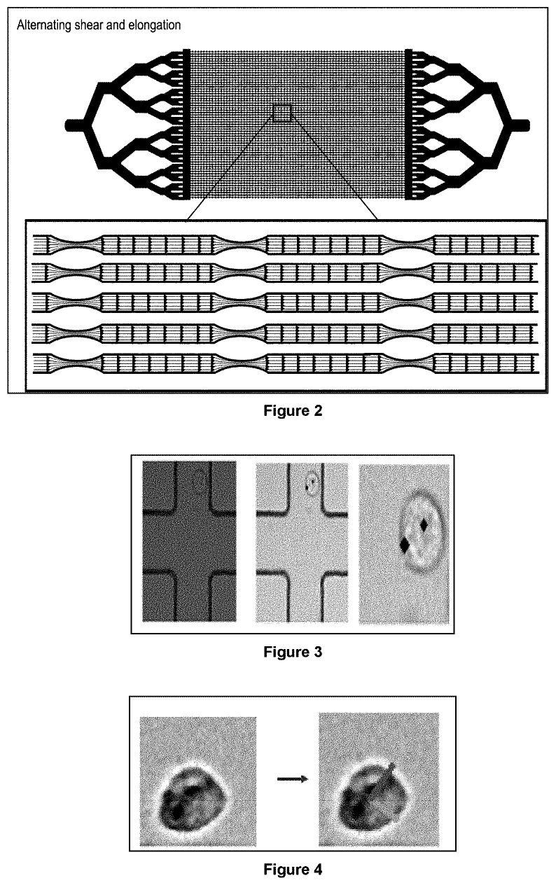

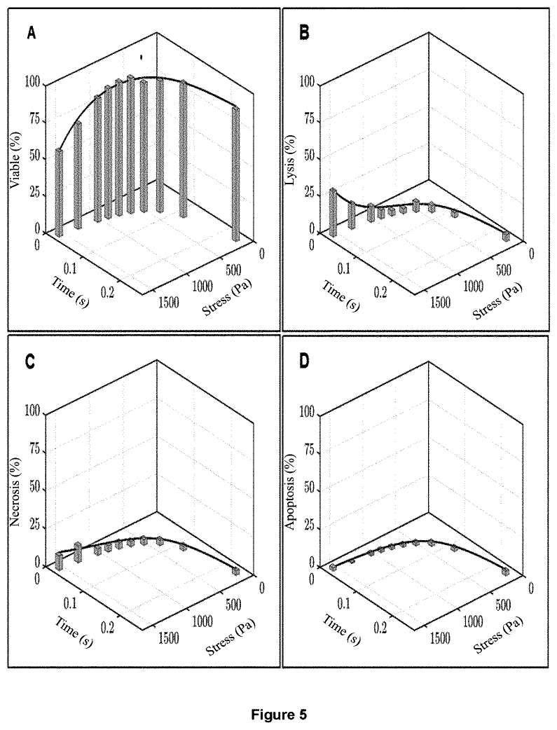

### Examples

### Example 1: Materials and Methods for Characterizing Cells after the Application of Physical Stress

AD-MSC cells are cultured in MSC-Growth culture medium, with a seeding concentration of around 2,500 cells per cm². The medium was changed every two days. To achieve 70% confluence, the cells were incubated for seven days in a T175 flask.

The fibroblast cells are cultured in DMEM GLUTAMAX-Gibco culture medium, with a seeding concentration of around 5,500 cells per cm². To achieve 80% confluence, the cells were incubated for seven days in a T175 flask.

The HEK293T cells are cultured in DMEM GLUTAMAX-Gibco culture medium, with a seeding concentration of around 12000 cells per cm². To achieve 80% confluence, the cells were incubated for four days in a T175 flask. During incubation, the temperature is maintained at 37° C. and the CO₂ concentration at around 5%.

To unhook the cells from the flask surface, first the culture medium was removed. The cells attached to the flask are rinsed with 15 ml PBS. Next, 5 ml Trypsin-EDTA 0.5% was added to the flask. Trypsin was applied for two minutes at 37° C., then 10 ml of culture medium containing fetal calf serum was added to the flask to stop the reaction. The suspension was placed in a tube and centrifuged at 1200 rpm/210 g for 5 minutes. The liquid was removed and the cells suspended in a solution of PBS and Ficoll at a concentration of one million per ml.

The microfluidic shear stress device consists of a PDMS microfluidic channel with a diameter of 50 micrometers and a length of 10 cm. The cells were suspended in a fluid (a solution of PBS and Ficoll) with a viscosity of 1.91 mPa·s.

The microfluidic device for elongational stress consists of a main microfluidic channel with a diameter of 162 micrometers and a length of 1 cm. The channel cross-section first decreases from 162 to 30 micrometers and then increases from 30 to 162 micrometers. This change in cross-section takes place over a distance of 360 micrometers. The viscosity of the suspending fluid (a solution of PBS and Ficoll with a volume concentration of 60% Ficoll) is 1.91 mPa·s.

Cell suspension and cell-free fluid are injected into the device using syringe pumps and 3 mm diameter PEEK tubing.

Cells are injected into the device at a flow rate of between 25 and 800 microliters per minute. Measurements and cell harvesting are carried out after hydrodynamic stability has been achieved within the system.

After passing through the stress zone, the cells were harvested. The suspension of harvested cells is centrifuged to remove the suspending fluid (Ficoll solution and PBS) and replace it with the labeling buffer.

For viability testing, cells are stained with annexin V, a marker for apoptotic cells, or propidium iodide, a marker for necrotic cells. To this end, A population of 100,000 cells corresponding to each injection rate was suspended in 100 microliters of labeling buffer. Then, 2 microliters of annexin V marker and 2 microliters of propidium iodide were added to the buffer. The suspension of cells with the viability markers was incubated for 15 minutes in a dark place. The cells were then centrifuged and rinsed with labeling buffer.

To test the stemness and differentiation state of the AD-MSC stem cells, the cells are labeled using the BD Human Mesenchymal Stem Cell Analysis Kit (BDB562245). The kit contains the hMSC-positive markers CD90, CD105, CD73 and CD 44 and the hMSC-negative markers CD34, CDIIb, CD19, CD45 and HLA-DR.

For the staining procedure, 100,000 cells are suspended in 100 microliters of BD Stain Buffer (FBS), then 5 microliters of each positive marker and 20 microliters of each negative marker are added to the buffer. The suspension of cells with markers was incubated for 15 minutes in a dark place. The cells were then centrifuged and rinsed with labeling buffer.

The cells are studied by cytometry (FACS Canto II) and characterized according to their state: viable cell, lysis, necrosis, apoptosis or stemness. Each measurement point corresponds to an analysis of at least 50,000 cells.

Based on the results obtained, a mathematical model is developed to predict the physiological state of the cells based on stress intensity and duration. The model is a mathematical expression that relates parameters, for example a polynomial expression, a power law, a sum of sines or other 2D or 3D mathematical expressions. For example, we can use a power law in the form y=ax^{k}+c, which establishes a relationship between x and y. In this model a is a constant of proportionality, k is the exponent and c is the error term. Using this method, we model the physiological state of cells before and/or after passage through the stress zone based on stress intensity and stress residence time. For this purpose, the model is established as follows: Physiological state=A×Stress^{B}+C; Residence time=D/Stress. Parameters A, B, C and D differ for each cell type, stress type and number of passages.

### Example 2: Characterization of the Viability State of AD-MSC Stem Cells after Application of Shear Stress

AD-MDC stem cells were cultured and subjected to shear stress of varying intensity and duration. After application of these stresses, the viability of the cells is characterized based on the intensity of the shear stress and the residence time of the cells under stress. Characterization of the physiological state (viable, lysis, necrosis or apoptosis) of the cells was carried out as described in Example 1.

The mathematical model defined in this case is as follows:

$$State = A \times {Stress}^{B} + C ; Time = D / Stress .$$

The values A, B, C and D as defined for each of the possible physiological states are as follows:

TABLE 1

| | |
|---|---|
| Viable | A = −4.009e−12, B = 4.046, C = 90.26, D = 30.6 |
| Lysis | A = 9.183e−13, B = 4.215, C = 4.172, D = 30.6 |
| Necrosis | A = 9.872e−08, B = 2.474, C = 2.918, D = 30.6 |
| Apoptosis | A = 43.1, B = −0.6166, C = 0.8814, D = 30.6 |

The results obtained are shown in the attached FIG. 5. These results show that human AD-MSC stem cells are sensitive to shear stress intensity and residence time under stress. We observe that AD-MSC cells have a mechanical capacity with respect to stress of around 1000 Pa with a residence time of around 0.25 s. Beyond these values, AD-MSC stem cells can no longer withstand the stress and suffer mortality. Mortality by lysis or necrosis is the most common pathway, while the level of apoptosis is negligible in this range of stresses and residence times.

### Example 3: Physiological State of Fibroblasts after the Application of Shear Stress

Fibroblast cells were cultured and cell samples subjected to shear stress of varying intensity and duration. After applying these constraints, the viability or lysis, necrosis or apoptosis of the cells was determined as described in Example 1.

The mathematical model defined in this case is as follows:

$$State = A \times {Stress}^{B} + C ; Time = D / Stress$$

The values A, B, C and D as defined for each of the possible physiological states are as follows:

TABLE 2

| | | |
|---|---|---|
| Viable | stress < 800 Pa | A = −3.105e−12, B = 5.288, C = 86.53, D = 30.6 |
| Viable | stress > 800 Pa | A = 1.342e+16, B = −4.928, C = 2.308, D = 30.6 |
| Lysis | stress < 800 Pa | A = 1.003e−15, B = 5.288, C = 7.159, D = 30.6 |
| Lysis | stress > 800 Pa | A = −1.304e+16, B = −4.928, C = 84.04, D = 30.6 |
| Necrosis | | A = 1.054e−14, B = 4.52, C = 5.476, D = 30.6 |
| Apoptosis | | A = −1.355^{e}+05, B = −1.881, C = 4.821, D = 30.6 |

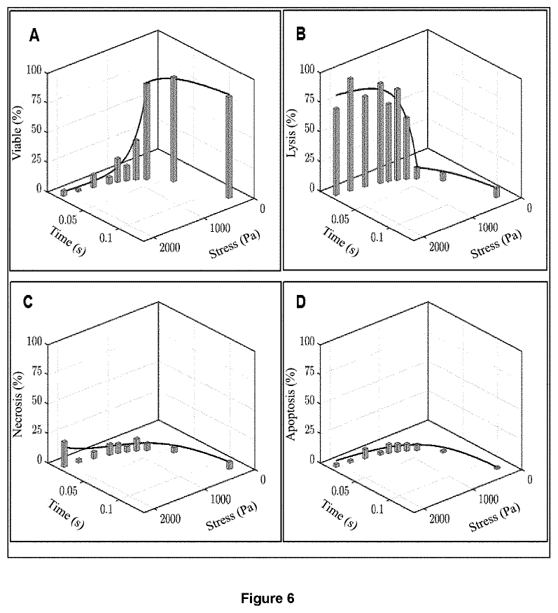

The results obtained are shown in the attached FIG. 6. These results show that fibroblast cells are highly sensitive to shear stress and residence time under stress. We can see that fibroblast cells up to 900 Pa and for 0.04 s are able to withstand the shear stress without being damaged, that is, without exhibiting lysis, apoptosis or necrosis. These values represent the mechanical capacity of fibroblast cells to withstand shear stress. However, beyond this mechanical capacity, the physiological response, that is, the loss of viability, of the fibroblasts is considerable. In fact, the fibroblasts do not withstand stress and the viability level of 85% at 900 Pa suddenly drops to 30% at 1000 Pa and reaches a level of less than 5% viability at 2000 Pa. Fibroblast cell mortality is caused more by the lysis pathway, and the other physiological states are negligible.

### Example 4: Differentiation State of AD-MSC Human Mesenchymal Stem Cells after Application of Shear Stress

AD-MSC stem cells were cultured as shown in Example 1. As a prerequisite, AD-MSC stem cells were characterized by cytometry to confirm their stemness prior to the application of shear stress of varying intensity and duration. After passing through the stress zone, the cells are harvested and resuspended in DMEM (+) culture medium. The differentiation state was characterized re-culturing the cells. Seeding was carried out at 80% confluence. After three weeks of incubation, cells were harvested, labeled with the BD Human Mesenchymal Stem Cell Analysis Kit (BDB562245) and analyzed by flow cytometry (FACS Canto II). Each measuring point corresponds to at least 30,000 cells. Characterization was performed using flow cytometry, which identifies the state of the cells after application of the stress in a binary fashion: differentiated or undifferentiated. The cytometer characterization procedure was carried out as shown in Example 1.
The mathematical model defined in this case is as follows: Undifferentiated state: Time=D/Stress where D=30.6 in the range 0.147 s <time <0.39 s and 78<stress <2082 Pa

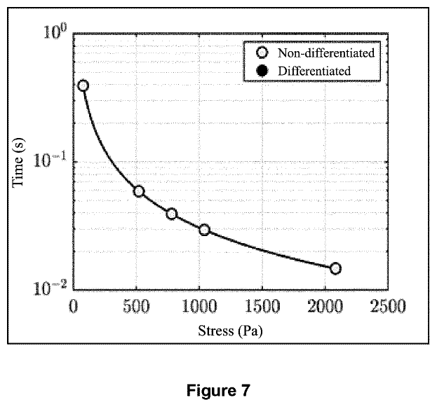

The results obtained are shown in the attached FIG. 7. These results show the absence of differentiated cells. This indicates that the AD-MSC human mesenchymal stem cells maintain their stemness after undergoing shear stress in the range shown in the figure. The physiological state of the AD-MSC cells, here the maintenance of stemness, was therefore unaffected by stress and residence time in the range 0-2100 Pa and 0.015-0.4 s.

### Example 5: Viability State of HEK293T Cells after Application of Elongation Stress

HEK293T cells were cultured and then cell samples were subjected to elongation stresses of varying intensity and duration. After applying these constraints, the viability or lysis, necrosis or apoptosis of the cells was determined as described in Example 1.

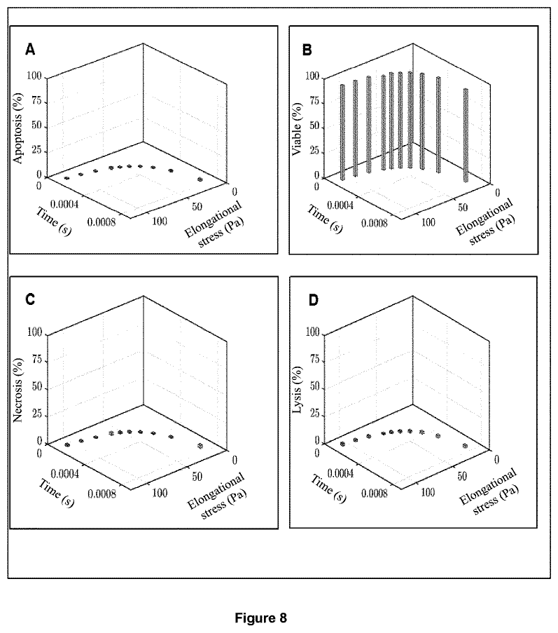

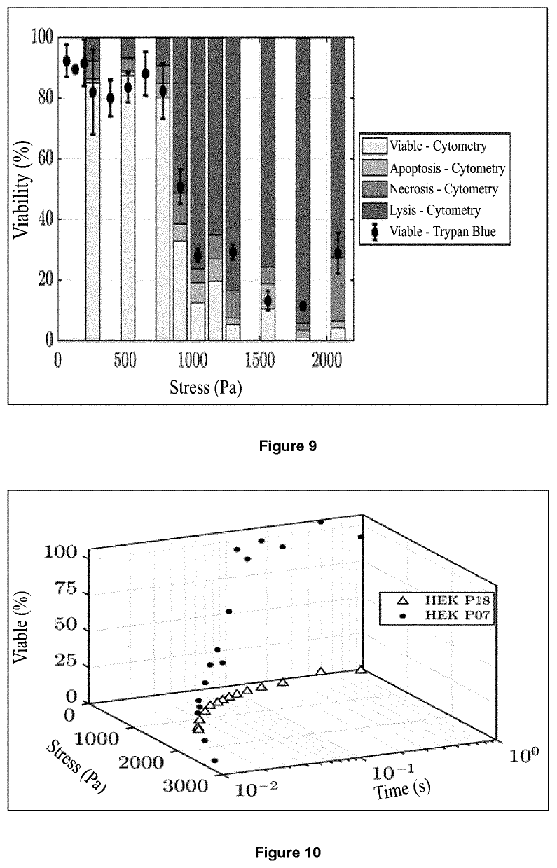

The results obtained are shown in the attached FIG. 8. These results show that the HEK293T cells have a high survival rate in the face of elongational stress in the stress range between 10 and 110 Pa and residence time between 0.1 ms and 0.8 ms. Lysis, necrosis and apoptosis are negligible compared to the viable state.

### Example 6: Viability State of Fibroblast Cells after Application of Shear Stress, as Measured by Two Protocols

Fibroblast cells were cultured and then cell samples were subjected to shear stresses of varying intensity and duration. After applying these stresses, cell viability was determined using two different protocols: cytometric analysis and trypan blue counting. Viability is shown based on shear stress intensity. The residence time under stress corresponds to the following equation:

$$time ( s ) = 30.6 / shear stress intensity ( Pa )$$

The cells were harvested after passage through the stress zone. They were then divided into two batches for cytometric and trypan blue analysis. For cytometric analysis, the batch was stained with Annexin V and propidium iodide. The marked cells were then analyzed by cytometer. Each measuring point corresponds to at least 50,000 cells. For trypan blue analysis, the batch of cells was mixed with trypan blue. Three counts were carried out over the entire surface of the Malassez counting chamber. The error bar corresponds to these three counts. The results obtained are shown in FIG. 6.

### Example 7: Viability State of HEK293T Cells after Application of Shear Stress

HEK293T cells were cultured and then cell samples were subjected to shear stress of varying intensity and duration, as described in Example 1. The HEK293T cells were harvested after passage through the stress zone, then mixed with trypan blue. Three counts were carried out over the entire surface of the Malassez counting chamber. The value corresponds to the average of these three counts.

The results obtained are shown in the attached FIG. 10. These results show that the number of cell passages in the cell culture clearly affects the mechanical capacity of the HEK293T cells. These cells with a passage P07 (7 passages) withstand stress perfectly, with a 100% viability rate for stresses below 200 Pa. Beyond this mechanical capacity, however, the same passage P07 HEK293T cells experience a drop in viability. However, the same cell line but with a P18 passage (18 passages) loses this mechanical capacity and cannot withstand the slightest stress.

### Example 8: Demonstrating Measurement Reproducibility

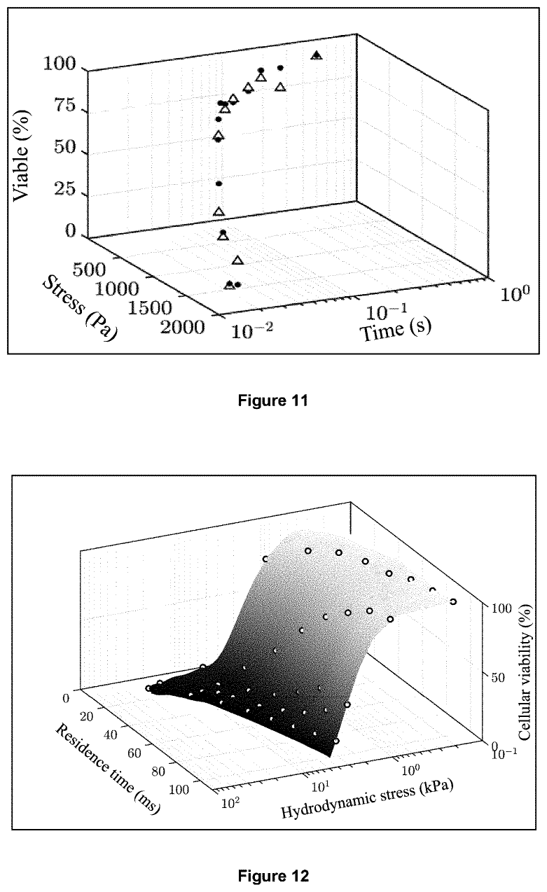

The characterization of the physiological state of fibroblast cells after application of shear stress was carried out in duplicate. Both measurements were done on the same cell sample (P06). The reproducibility of the experiment has an average error of 3.45% viability. The analyses performed by the system are therefore reproducible. The results obtained are shown in the attached FIG. 11.

### Example 9: Mechanical Capacity of Cells Based on Stress Intensity and Stress Residence Time

Residence time is defined as the period during which cells are exposed to hydrodynamic stress. In a microfluidic device and method according to the invention, stress is maintained at a constant intensity, while dwell time depends on the flow rate and viscosities wherein the cells are suspended. This approach makes it possible to decouple the two parameters and study their effect independently. Fibroblasts were suspended in fluids with viscosities of 1, 5, 10, 15 or 20 mPa·s and injected through the microfluidic capillary tube at flow rates ranging from 117 to 942 μl/min. This configuration covered a shear stress intensity range of 0.16 to 25.59 kPa and a dwell time of 12.5 to 100.7 ms.

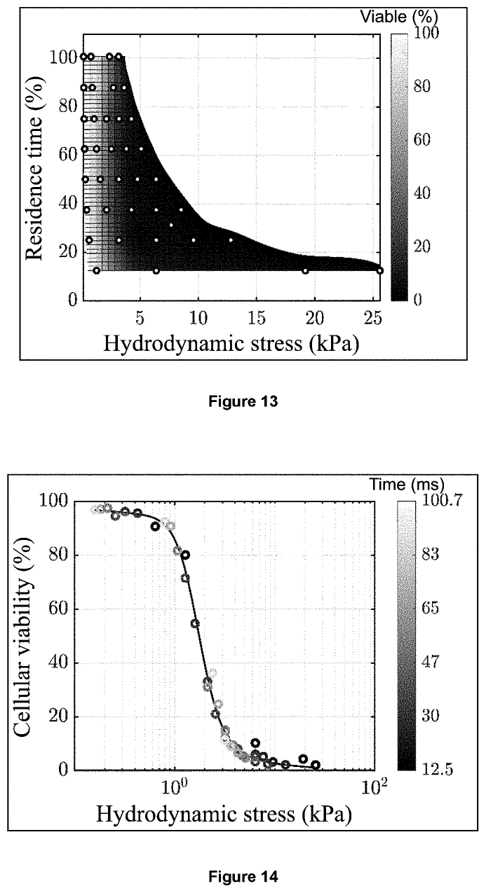

FIG. 12 and FIG. 13 show a mapping of fibroblast cell viability as a function of stress intensity and dwell time. These two figures show the same data with 3D and 2D views and linear and logarithmic scales. The white dots are the experimental data and the surface represents the mathematical model fit. Within the ranges indicated, for a given dwell time, increasing the stress intensity reduces cell viability. However, for a constant stress intensity, cell viability is independent of dwell time. FIG. 14 shows fibroblast viability only as a function of stress intensity, while shades of grey identify dwell time. Owing to the three representations of the same data, it can be concluded that the viability and mechanical capacity of the fibroblasts do not depend on residence time in the range 12.5 to 100.7 ms.

These results indicate that, in this case, fibroblast viability can only be characterized based on stress intensity.

In a method according to the invention, stress intensity and residence time are totally decoupled. It is possible to keep one of these parameters constant and vary the other. In this way, it is possible to gain a detailed insight into the effect of residence time and shear stress intensity as two independent parameters.

### Example 10: Device Reproducibility and Precision

To ensure reproducibility of experiments and results, the inventors measured the mechanical capacity of Madin-Darby Canine Kidney (MDCK) cells 6 times. To eliminate the biological variability that will affect the assessment of machine and procedure performance errors, a population of cells was divided into 6 batches. Mechanical capacity was measured on each cell batch. Cells were exposed to shear stresses of 0.175, 0.469, 2.716 and 6.397 kPa, then their viability was measured by cytometer. Standard deviation, standard error and coefficient of variation were calculated using the following formulas:

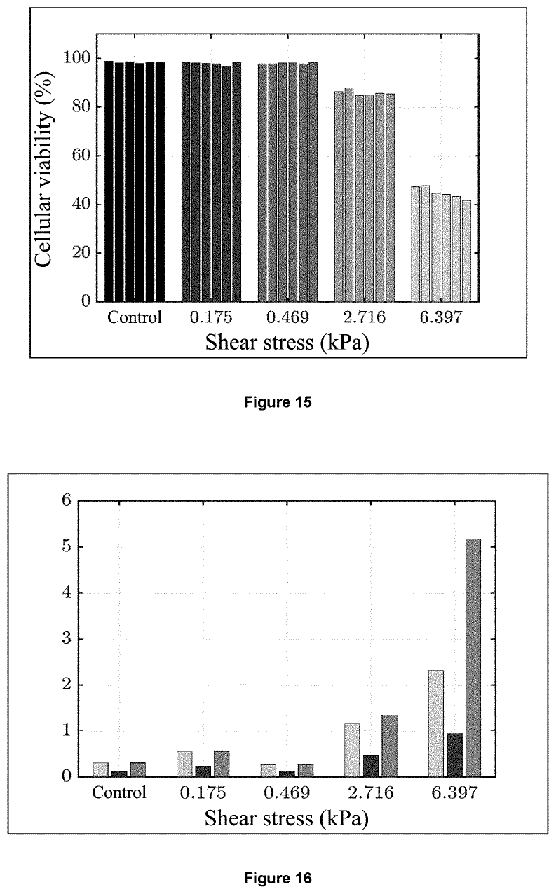

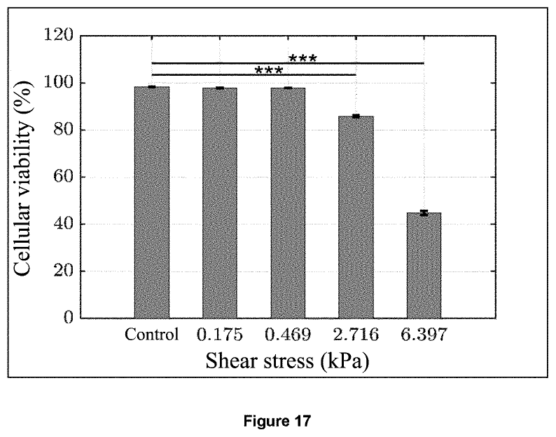

The results show that the standard error for the 6 trials is 1% lower in the worst case. This example perfectly shows the precision and performance of the machine, and the procedure that leads to reliable and very fine measurements. This precision allows advanced analysis with a high degree of reliability, as shown in FIGS. 15 to 17.

### Example 11: Mechanical Capacity of Different Cell Lines

With a device according to the invention, it is possible to measure the mechanical capacity against hydrodynamic stresses of different cells: primary cell line, immortalized cell line, stem cells, etc. The mechanical capacity of the following cell lines was analyzed: Adipose tissue-derived mesenchymal stem cell (AD-MSC), Wharton's jelly-derived mesenchymal stem cells (WJ-MSC), Bone marrow-derived mesenchymal stem cell (BM-MSC), human dermal fibroblasts, Human Umbilical Vein Endothelial Cells (HUVEC), Cancer-associated fibroblasts (CAF), Human embryonic kidney 293T cells (HEK293T) and Madin-Darby canine kidney (MDCK).

Cells were suspended in two fluids with viscosities of 1 and 5 mPa·s and injected through a 50 μm diameter microfluidic channel with a flow rate ranging from 85 to 942 μl/min and subjected to a shear stress of 0.11 to 6.4 kPa. Collected cells were stained with propidium iodide and analyzed by cytometry to quantify the necrotic cell population induced by shear stress. To confirm that the cells are only damaged by shear stress during injection into the microfluidic channel and not by static syringe pressure, fibroblast cells were injected without passing through the microfluidic channel and analyzed by cytometry. We confirmed that cell viability was unchanged compared to the control cell population viability of 97%. Moreover, in the case of adherent cells, special care was taken during the experiment to limit their death due to suspension conditions. Additionally, the time between cell harvesting and exposure to shear stress was just 5 min, and identical for all cell lines.

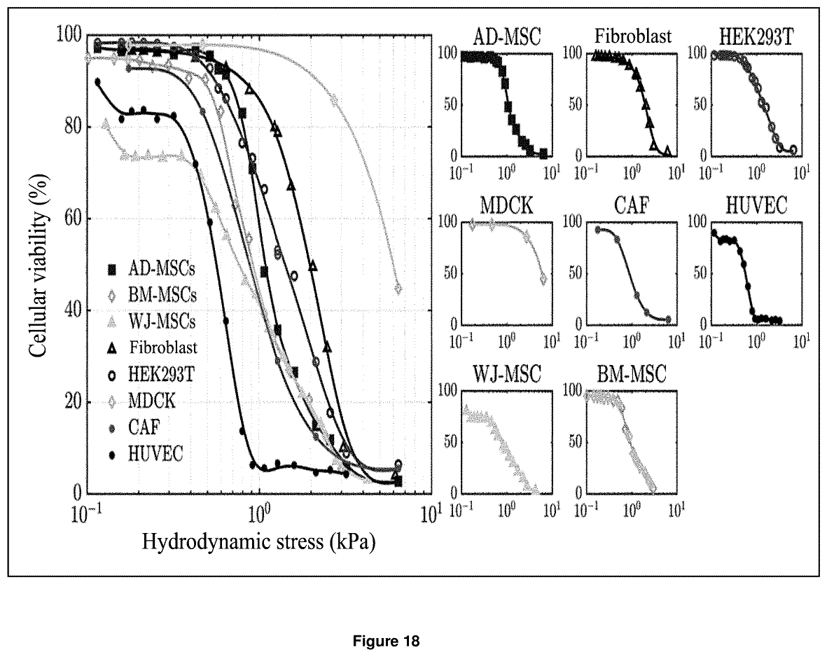

FIG. 18 shows the viability of cell lines after the application of shear stress. The dots are experimental data and the lines are fitting curves. Such a cell viability evolution profile can be described as having two phases. Firstly, cell viability shows a plateau up to a certain shear stress limit, that is, the ability of cells to withstand shear stress. Then, after shear stress, which corresponds to the cell's mechanical capacity, cell viability deteriorates, dropping to less than 5% of viable cells. Before the shear stress linked to the mechanical capacity of each cell, viability varies by less than 5% compared with the non-sheared control cell population. It is also worth noting that while all the cell types studied show similar trends, each cell line has a specific sensitivity to shear stress as a mechanical capacity, AD-MSC: 0.64 kPa, WJ-MSC: 0.35 kPa, BM-MSC: 0.48 kPa, fibroblasts: 0.64 kPa, HUVEC: 0.32 kPa and HEK293T: 0.43 KPa.

### Example 12: Evolution of Mechanical Capacity with Cell Aging and Incubation Time

To explore the link between cellular aging and mechanical capacity, fibroblasts were subcultured (passaged) once a week and subjected to shear stress. The experiments were continued for four passages, from P07 to P10. The passage is the process of sub-culturing animal cells. “P07” refers to the 7th generation of cell passage in culture. Subculture practices were strictly identical between the different passages. The viability of fibroblast cells after being subjected to several intensities of shear stress was measured for each passage number by flow cytometry. Cells were suspended in fluids with viscosities of 1 and 5 mPa·s, then exposed to shear stress between 0.159 and 6.14 kPa. Flow rates ranged from 117 to 942 μl/min for both suspension fluids.

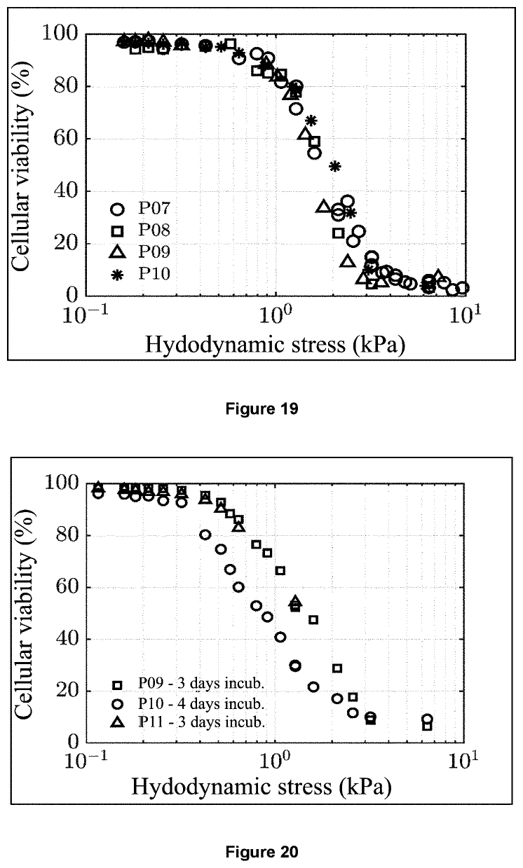

FIG. 19 shows that sequential subculture practices do not affect the mechanical capacity of fibroblasts in the face of shear stress. In fact, they have a similar mechanical capacity regardless of the passage number. Up to the shear stress corresponding to the cell's mechanical capacity of 0.577 kPa, fibroblast viability is unaffected (96%), while control viability is 97%. In the range from 0.639 kPa to 4.78 kPa, viability deteriorates sharply from 92% to 5%, reaching a plateau below 5%.

A similar study was applied to HEK293T cells. Unlike fibroblasts, HEK293T cells were subcultured twice a week from passage number P09 to P11. As a result, the duration of cell growth alternates between 3 and 4 days between successive subcultures.

Cells were suspended in fluids with viscosities of 1 and 5 mPa·s, then exposed to shear stress in the range 0.11 to 6.4 kPa. Flow rates ranged from 85 to 942 μl/min for both suspension fluids. Cell viability was analyzed by cytometry.

As shown in FIG. 20, the mechanical capacity of HEK293T cells is affected by variation in the incubation period for successive cell subcultures. The incubation period before applying the stress and before measurement is 3 or 4 days. HEK293T cells with a 3-day incubation period have a mechanical capacity of 0.516 kPa, while HEK293T cells with a longer incubation period have a reduced mechanical capacity of 0.319 kPa.

### Example 13: Repeating a Series of Stresses

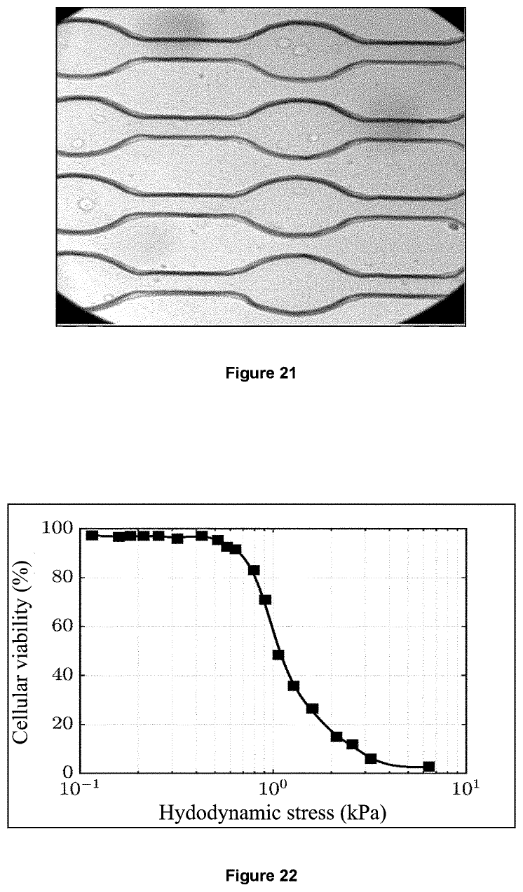

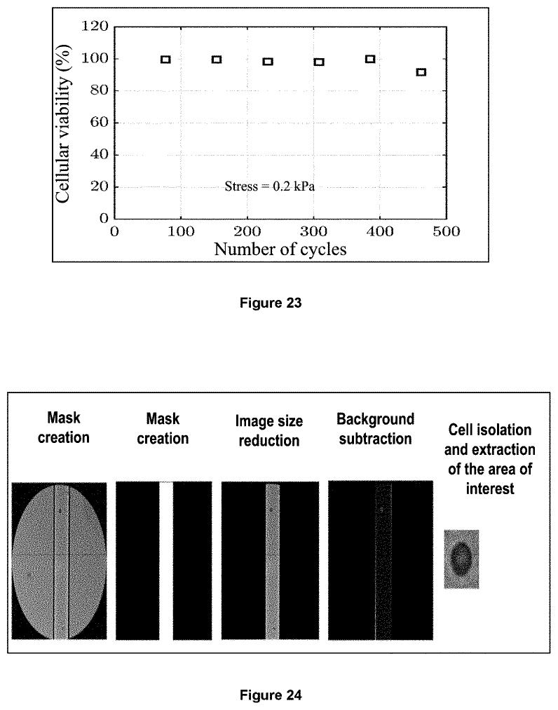

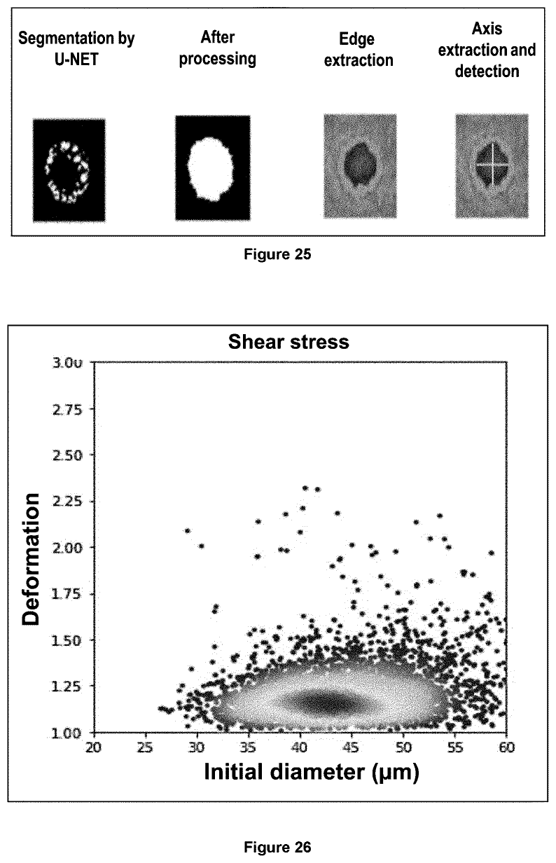

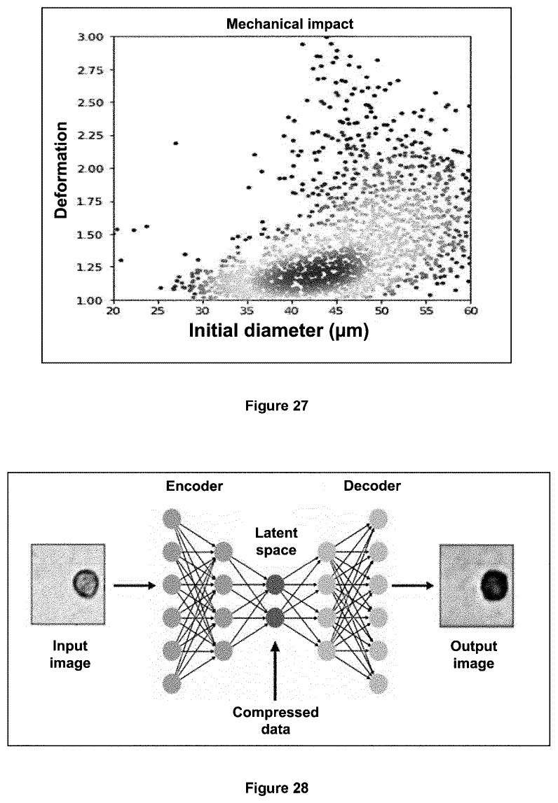

To explore the effect of repeated identical stresses on the cell's mechanical capacity, AD-MSCs cells were suspended in a fluid with a viscosity of 1 mPa·s and exposed to repeated hydrodynamic stresses. This type of stress is defined as a cyclic succession of the same stress. FIG. 21 shows this sequence. The cells are subjected to shear stress, enter a shear-free zone and are then sheared again. This cycle was carried out for a batch of cells, with a number of cycles equal to 77, 154, 231, 308, 385 and 462, at a stress of 0.2 kPa. This stress is less than the mechanical capacity of the cells, 0.6 kPa, as shown in FIG. 22. After exposure to different numbers of cycles, the cells were analyzed by flow cytometer to determine cell viability. FIG. 23 shows cyclic stress below the mechanical capacity of the AD-MSC cell, without affecting cell viability.

### Example 14: Cell Localization and Isolation

Cell localization and isolation are performed using an algorithm based on the original images taken by the high-speed camera. Firstly, a mask is created to reduce the size of the original image by superimposition. Secondly, image processing functions such as erosion and dilation are applied to reduce image background noise and reveal cell localization with greater contrast. Isolation is then performed by defining the region of interest (ROI) (FIG. 24).

### Example 15: Positioning and Measuring Minor and Major Axes

The positioning and measurement of minor and major axes is performed using a supervised learning model. The first step is to apply the supervised model (in this case U-Net) to the resulting cell photo in FIG. 24. This identifies the size and shape of the cell. Secondly, an “edge extraction” type image processing function is performed in order to position the cell contour on the image taken from FIG. 24. Then, the minor and major axes are measured, the major axis being the largest diameter and the minor axis being the smallest diameter. These results can be displayed in a graph showing the level of deformability based on cell size. Here, our results demonstrate the accuracy of our image processing deformability measurements for shear and crash tests.

### Example 16: Image Quality Improvement Using an Unsupervised Model

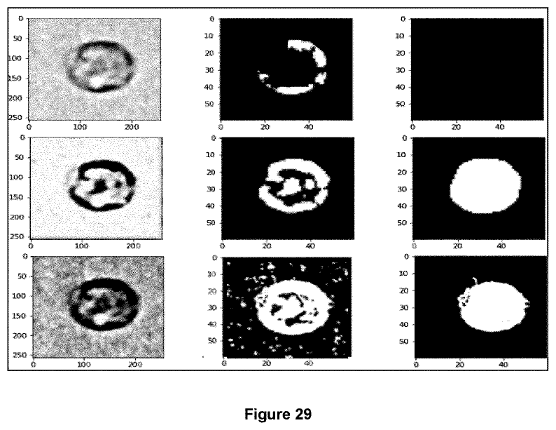

The images resulting from the cell localization and isolation steps are not always of good quality for measuring major and minor axes. This is because these images are sufficiently noisy to prevent the supervised model from segmenting the cell. In order to improve this image quality, the application of an unsupervised model is necessary. Here, it is shown that the application of an “autoencoder” neural network can reconstruct the image resulting from ROI extraction with better quality (FIG. 28). FIG. 29 demonstrates the relevance of this approach. In FIG. 29, line 1 shows results without autoencoder application, line 2 shows results with variational autoencoder application, and line 3 shows results with denoising autoencoder application).

### Example 17: Improving Cell Detection and Deformability Measurements by Coupling an Unsupervised and a Supervised Model

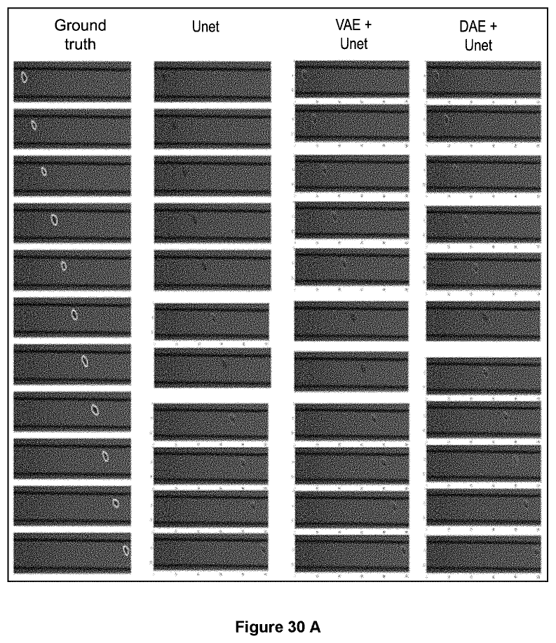

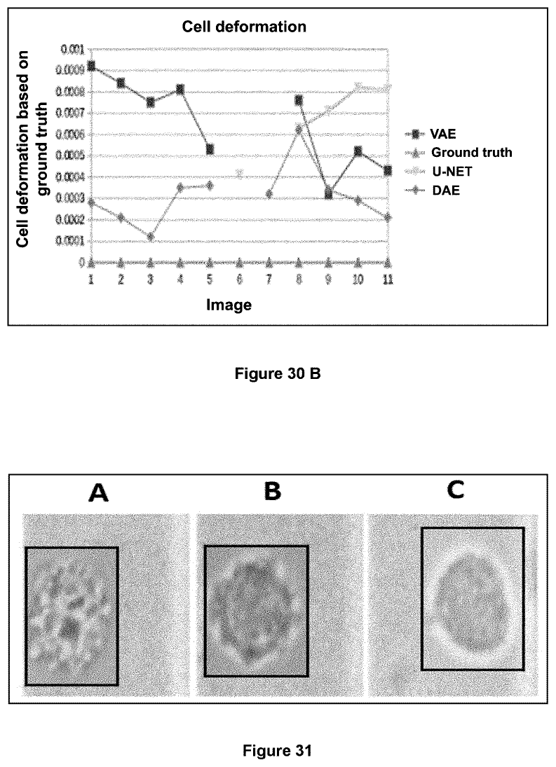

The coupling of an unsupervised and a supervised model can be used to improve the detection of cells within an image, as well as its deformability measurement. In this example, we demonstrate this approach by coupling autoencoder and U-Net neural networks to images taken from shear measurements (FIG. 30A).

The numerical values are as follows (Table 3):

TABLE 3

| | U-NET | VAE + U-NET | DAE + U-NET |
|---|---|---|---|
| Precision | 0.59 | 0.78 | 0.81 |
| Recall | 0.63 | 0.69 | 0.71 |
| F1-score | 0.61 | 0.75 | 0.77 |

We can observe that the application of the supervised network (U-Net) alone does not enable detection and tracking of all cell kinematics, whereas coupling with an unsupervised model (VAE or DAE autocoder) improves detection and measurement (higher Precision, Recall and F& score) (FIG. 30B).

Thus, the measurement improvement is described by subtracting the deformability measurement from a standard manual approach. That is, each image has been processed by the user without image processing or the application of a learning model.

### Example 18: Prediction of a Cell's Physiological State by a Classification Model Using Supervised Learning

A supervised learning model can be used to classify the cell's physiological state by image analysis. This example shows the classification of 3 cells annotated as necrotic (A), apoptotic (B) and living (C).

1. A device for applying at least one hydrodynamic stress of determined intensity and duration to at least one cell suspended and moving in said device, and for characterizing said cell, during the application of said stress and optionally for characterizing said cell after the application of said stress, the device comprising:
a microfluidic circuit comprising at least one segment, said segment comprising at least: i) a main channel configured for the circulation of a fluid containing said cell in suspension, ii) a fluid inlet and a fluid outlet, iii) means for introducing and establishing a flow of said fluid within said channel, and
means for characterizing said cell during the application of said stress, characterized in that said at least one segment is configured for the application to said cell of at least one hydrodynamic stress selected from: hydrodynamic compressive stress, hydrodynamic shear stress and hydrodynamic extensional stress, and for the optional application of at least one mechanical impact to said cell.

2. The device according to the preceding claim, wherein the characterization of said cell has as its object at least one criterion selected from: size, shape, appearance of the outer membrane, appearance of the cytoplasm, presence of at least one marker on the cell surface, protein content of the cell and nucleic acid content of the cell.

3. The device according to one of the preceding claims, characterized in that said at least one segment A, configured for the application to said cell of a compressive stress, comprises, or consists of, a first channel joined, at the same level, by two channels, each making an angle of between 30 and 150 degrees with said first channel.

4. The device according to one of the preceding claims, characterized in that said at least one segment B, configured for the application to said cell of a shear stress, comprises, or consists of, a channel with a diameter of between 10 and 2000 μm, preferably between 20 and 200 μm.

5. The device according to one of the preceding claims, characterized in that said at least one segment C, configured for the application to said cell of an extensional stress, comprises, or is constituted by, i) a channel of increasing or decreasing cross-section or ii) a first channel joined by a second channel wherein the fluid circulation takes place in a direction different from, preferably opposite to, that of the first channel.

6. The device according to one of the preceding claims, characterized in that said at least one segment D, configured for the application to said cell of a mechanical impact, comprises, or consists of, a first channel within which the streamline of the cells encounters an obstacle, such as in particular the wall of a second channel.

7. The device according to any one of the preceding claims, characterized in that said circuit comprises: at least one segment A and/or at least one segment B and/or at least one segment C, and optionally at least one segment D, combined with each other.

8. The device according to any one of the preceding claims, characterized in that said circuit is configured for the application to said cell of at least one sequence comprising, or consisting of, at least two successive physical stresses of different natures.

9. The device according to any one of claims 1 to 7, characterized in that said circuit is configured for the application to said cell of at least one sequence comprising, or consisting of, at least two successive hydrodynamic stresses of the same nature, and optionally at least two successive mechanical stresses of the same nature.

10. The device according to any one of the preceding claims, characterized in that the intensity of at least one hydrodynamic stress applied to the cell is between 10⁻³ kPa and 10⁵ kPa, preferably between 10⁻³ kPa and 10⁴ kPa, preferably between 0.1 and 10³ kPa, preferably between 1 and 10² kPa and/or in that the total duration of the application of said at least one hydrodynamic stress is between 1 μs and 10000 s, preferably between 1 μs and 100 s, preferably between 1 μs and 1 s, preferably 10 μs and 100 ms.

11. The device according to any one of the preceding claims, characterized in that the characterization of said cell after the application of said at least one stress is carried out for a period of between 0 and 120 days, preferably between 0 and 30 days, preferably between 0 and 1 day after the application of said at least one physical stress.

12. The device according to any one of the preceding claims, characterized in that the characterization of said cell during the application of said at least one stress comprises, or consists of, at least one discrete characterization or at least one characterization carried out over a duration of between 1 μs and 10000 s, preferably 1 μs and 100 s, preferably between 1 us and 1 s, preferably 10 μs and 100 ms.

13. The device according to any one of the preceding claims, characterized in that said means for characterizing said at least one cell is selected from: a cytometer, a microscope, means for analyzing the protein content of the cell, means for analyzing and sequencing the nucleic acids of said cell, and image capture means combined with image analysis means.

14. The device according to the preceding claim, characterized in that said image analysis means comprises or consists of: a central computer unit comprising software means adapted for image analysis.

15. The device according to one of claim 13 or 14, characterized in that said image analysis means further comprises a first classification model, previously trained with a training dataset, and comprising a supervised, unsupervised or semi-supervised machine learning algorithm, said first classification model being adapted for predicting the physiological state of a given cell from characteristics of said cell.

16. The device according to one of claims 13 to 15, characterized in that said image analysis means further comprises a second classification model, previously trained with a training dataset, and comprising a supervised, unsupervised or semi-supervised automatic learning algorithm, said second classification model being adapted for detecting and monitoring the deformation of a given cell, in response to at least one physical stress.

17. A method for applying at least one physical stress of determined intensity and duration to at least one cell in suspension, and for characterizing said cell during said application, and optionally after said application, the method comprising the following steps:
a) depositing and circulating a fluid containing said at least one cell in suspension in a microfluidic circuit comprising at least one segment, said segment comprising at least: i) a main channel configured for the circulation of a fluid containing said cell, ii) a fluid inlet and a fluid outlet, iii) means for introducing and establishing a flow of said fluid within said channel, and
b) characterizing said cell during the application of said at least one stress; and optionally after the application of said at least one stress, characterized in that said at least one segment is configured for the application to said cell of at least one hydrodynamic stress selected from: hydrodynamic compressive stress and/or a hydrodynamic shear stress and/or a hydrodynamic extensional stress, and optionally at least one mechanical impact.

18. The method according to the preceding claim, wherein the characterization of said cell has as its object at least one criterion selected from: size, shape, appearance of the outer membrane, appearance of the cytoplasm and presence of at least one surface marker.

19. The method according to one of claim 17 or 18, wherein the cell is subjected to:
at least one shear stress of intensity between 10⁻³ kPa and 10⁵ kPa, preferably between 10⁻³ kPa and 10⁴ kPa, preferably between 0.1 kPa and 10³ kPa and/or for a duration between 1 μs and 10,000 s, preferably between 1 μs and 100 s, preferably between 10 μs and 1 s, preferably between 10 μs and 10 ms, and/or
at least one compressive stress of intensity between 10⁻³ kPa and 10³ kPa, preferably between 10⁻³ kPa and 10² kPa, preferably between 10⁻³ kPa and 10 kPa and/or for a duration between 1 μs and 10 s, preferably between 1 μs and 1 s, preferably between 10 μs and 10 ms and/or
at least one extensional stress of intensity between 10⁻³ kPa and 10³ kPa, preferably between 10⁻³ kPa and 10² kPa, preferably between 10⁻³ and 10 kPa and/or for a duration between 1 μs and 10 s, preferably between 1 μs and 1 s, preferably between 10 μs and 10 ms,
and optionally at least one mechanical impact of intensity between 1 and 300 m/s and/or for a duration of less than 1 μs.

20. The method according to one of claims 17 to 19, characterized in that it further comprises a step of predicting the physiological state of a cell by a previously trained first classification model, based on the characteristics determined in step b).

21. The method according to one of claims 17 to 20, characterized in that it further comprises a step of predicting the physiological state of a cell by a first previously trained classification model, characterized in that said first classification model comprises: a machine learning algorithm, a supervised learning neural network or a multi-class probabilistic classification algorithm, previously trained with a training dataset.

22. The method according to one of claims 17 to 21, characterized in that it further comprises a step of monitoring the deformability of a cell at different times during its residence in said microfluidic channel, by a second previously trained classification model, based on the characteristics determined in step b).

23. A classification model, previously trained on a training dataset, for predicting, in a method according to any one of claims 17 to 22, a physiological state of a cell during and/or after the application of at least one hydrodynamic stress.

24. A use of a device according to one of claims 1 to 16, or of a method according to one of claims 17 to 22 or of a classification model according to claim 23 to characterize cells of the following type: prokaryotic cell, eukaryotic cell, animal cell, plant cell, human cell, stem cell, epithelial cell, fibroblast, blood cell, genetically modified cell or synthetic cell mimic.

25. A use of a device according to one of claims 1 to 16, a method according to one of claims 17 to 22 or a classification model according to claim 23, to determine the capacity of a cell.

26. A use of a device according to one of claims 1 to 16, a method according to one of claims 17 to 22 or a classification model according to claim 23 for defining at least one parameter of a bioprocess.

27. The use according to claim 26, characterized in that said bioprocess is selected from: bioprinting, cell therapy and bioproduction.
